(19) United States
(12) Patent Application Publication
(10) Pub. No.: US 2006/0263892 A1
(43) Pub. Date: Nov. 23, 2006

(54) Detection of ortho-diamine residues in analytes containing residual cyclizing agents

(75) Inventors: Gregory J. Withbroe, Gales Fery, CT (US); Megan McMahon, Centerbrook, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/410,561

(22) Filed: Apr. 24, 2006

Related U.S. Application Data
(60) Provisional application No. 60/678,075, filed on May 4, 2005.

(51) Int. Cl. G01N 33/00

(52) U.S. Cl. 436/106

(57) ABSTRACT

The present invention relates to a novel method for the analytical determination of an aromatic ortho diamine contaminant in a pharmaceutical compound or intermediate thereof prepared by treating said aromatic ortho diamine with a cyclizing agent. The method comprises the steps of: (i) treating the pharmaceutical compound or intermediate thereof containing the contaminant with acetone to form a 2,2,4-trimethyl-1,5-benzodiazepine derivative of the aromatic ortho diamine contaminant, (ii) determining the quantity of the 2,2,4-trimethyl-1,5-benzodiazepine derivative in the pharmaceutical compound or intermediate thereof, preferably by HPLC and (iii) correlating the quantity of the 2,2,4-trimethyl-1,5-benzodiazepine derivative with the quantity of aromatic ortho diamine contaminant originally present in the pharmaceutical compound or intermediate thereof. Formation of the 2,2,4-trimethyl-1,5-benzodiazepine derivative occurs rapidly under mild conditions thereby eliminating interference by residual cyclizing agent and enabling detection of aromatic ortho diamines at ppm levels.

### BACKGROUND OF THE INVENTION

This invention relates to a method for the analytical detection and determination of low levels of aromatic ortho diamines that are potential contaminants of pharmaceutical compounds and intermediates that are precursors to such pharmaceuticals.

Ortho aromatic diamines are commonly treated with cyclizing agents to form hetero-ring structures in the preparation of pharmaceuticals and pharmaceutical intermediates. For example Coe, et al in WO99/35131 (U.S. Pat. No. 6,410,550 B1) describe compounds that bind to neuronal nicotinic acetylcholine specific receptor sites and are useful in modulating cholinergic function that are derived from aromatic ortho diamines. These compounds are useful in the treatment of inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amylotropic lateral sclerosis (ALS), cognitive dysfunction, hypertension, bulimia, anorexia, obesity, cardiac arrythmias, gastric acid hypersecretion, ulcers, pheochromocytoma, progressive supramuscular palsy, chemical dependencies and addictions (eq, dependencies on, or addictions to nicotine (and/or tobacco products), alcohol, benzodiazepines, barbituates, opioids or cocaine), headache, stroke, traumatic brain injury (TBI), obsessive-compulsive disorder, psychosis, Huntington's Chorea, tardive dyskinesia, hyperkinesia, dyslexia, schizophrenia, multi-infarct dementia, age related cognitive decline, epilepsy, including petit mal absence epilepsy, senile dementia of the Alzheimer's type (AD), Parkinson's disease (PD), attention deficit hyperactivity disorder (ADHD) and Tourette's Syndrome.

Such pharmaceuticals and the pharmaceutical intermediates used in their preparation may contain residues of ortho aromatic diamines which even at low ppm levels are undesirable due to potential genotoxicity. The ability to analyze pharmaceuticals and their precursors for ortho aromatic diamines even at such low levels is therefore critical.

This task is often complicated by the presence of residual cyclizing agents within the pharmaceutical or precursor which obscure the actual quantity of aromatic ortho-diamines present by reacting with them to form more pharmaceutical or precursor.

One method to overcome such interference with the assay matrix is to derivatize the analyte in order to prevent further reaction with residual cyclizing agent during the analysis. Conventional derivatization techniques, such as N-acylations, N-alkylations, and cyclization (under basic or acidic conditions), failed to produce useful derivatives of 1-(4,5-diamino-10-aza-tricyclo[6.3.1.0^{2,7}]dodeca-2(7),3,5-triene-10-yl)-2,2,2-trifluoro-ethanone. This was primarily due to the presence of basic or acidic reactants interfering with HPLC analysis.

There is therefore a need for another method of derivatization, which will minimize or eliminate potential side reactions and which is compatible with analytical methods such as HPLC. The known reaction of aromatic ortho-diamines with 2 moles of acetone to form the corresponding 2,2,4-trimethyl-1,5-benzodiazepine (G. Kaupp, U. Pogodda, J. Schmeyers; Chem. Ber; EN; 127;11, 1994; 2249-2262; H. Morales, A. Bulbarela, R. Contreras; Heterocycles, EN; 24;1, 1986; 135-139; M. Pozarentzi, J. Stephanidou, C. Tsoleridis; Tetrahedron Lett.; EN; 43; 9; 2002; 1755-1758) is such a method and provides the basis for the analytical method of the present invention.

### SUMMARY OF THE INVENTION

The present invention provides a method for the analytical determination of an aromatic ortho diamine contaminant in a pharmaceutical compound or intermediate thereof, comprising the steps of: (i) treating the pharmaceutical compound or intermediate thereof containing the contaminant with acetone to form a 2,2,4-trimethyl-1,5-benzodiazepine derivative of the aromatic ortho diamine contaminant, (ii) determining the quantity of the 2,2,4-trimethyl-1,5-benzodiazepine derivative in the pharmaceutical compound or intermediate thereof, and (iii) correlating the quantity of the 2,2,4-trimethyl-1,5-benzodiazepine derivative with the quantity of aromatic ortho diamine contaminant originally present in the pharmaceutical compound or intermediate thereof.

The present invention also provides a method for the analytical determination of an aromatic ortho diamine contaminant in a pharmaceutical compound or intermediate thereof wherein the pharmaceutical compound or intermediate thereof contains residues of a cyclizing agent.

The present invention also provides a method for the analytical determination of an aromatic ortho diamine contaminant in a pharmaceutical compound or intermediate thereof wherein the aromatic ortho diamine contaminant has the formula

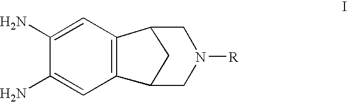

wherein R is H, COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 fluoro or chloro atoms; benzyl or t-butoxycarbonyl (t-Boc);

and the 2,2,4-trimethyl-1,5-benzodiazepine derivative formed in step (i) of the above procedure has the formula

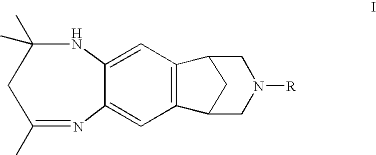

wherein R is H, COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 fluoro or chloro atoms; benzyl or t-butoxycarbonyl (t-Boc).

The present invention also relates to a method for the analytical determination of an aromatic ortho diamine contaminant in a pharmaceutical compound or intermediate thereof wherein R is —C(═O)CF₃ in the compound of formula II and the compound of formula I.

The present invention also relates to a method for the analytical determination of an aromatic ortho diamine contaminant in a pharmaceutical compound or intermediate thereof wherein a 2,2,4-trimethyl-1,5-benzodiazepine derivative of the aromatic ortho diamine contaminant is formed in the presence of a catalytic amount of strong acid such as hydrochloric acid.

The present invention also provides a method for the analytical determination of an aromatic ortho diamine contaminant in a pharmaceutical compound or intermediate thereof wherein the quantity of the 2,2,4-trimethyl-1,5-benzodiazepine derivative is determined by a chromatographic method such as HPLC.

The present invention also provides a process for the preparation of a compound of the formula

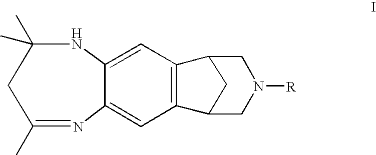

wherein R is H, COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 halo atoms, preferably with from 1 to 3 fluoro or chloro atoms; benzyl or t-butoxycarbonyl (t-Boc);

from an aromatic ortho diamine of the formula

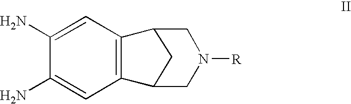

wherein R is H, COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 halo atoms, preferably with from 1 to 3 fluoro or chloro atoms; benzyl or t-butoxycarbonyl (t-Boc);

wherein the compound of formula II is treated with at least two molar equivalents of acetone and wherein the compound of formula I is useful in the analytical determination of the compound of formula II.

The present invention also relates to a compound of the formula

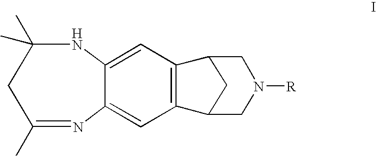

wherein R is H, COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 fluoro or chloro atoms; benzyl or t-butoxycarbonyl (t-Boc) which is useful in the analytical determination of the corresponding aromatic ortho diamine.

The present invention also relates to a compound of the formula

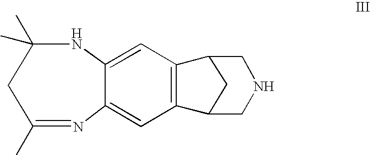

wherein said compound is useful in the detection of ortho aromatic diamine impurities in pharmaceuticals.

The present invention also provides a process for the preparation of a compound of formula

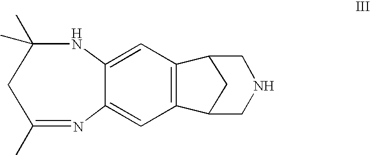

wherein a compound of the formula

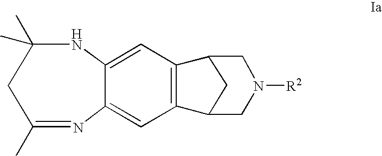

wherein R² is a protective group selected from COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 fluoro or chloro atoms; or t-butoxycarbonyl (t-Boc), is treated with an aqueous base.

The present invention also relates to a process for the analytical determination of residues of 1-(4,5-diamino-10-aza-tricyclo[6.3.1.0^{2,7}]dodeca-2(7),3,5-triene-10-yl)-2,2,2-trifluoro-ethanone, that is, the compound of formula II wherein R is trifluoroacetyl, in pharmaceuticals and pharmaceutical intermediates by formation of the corresponding 2,2,4-trimethyl-1,5-benzodiazepine derivative by reaction with two moles of acetone under nearly neutral conditions, using only a catalytic amount of aqueous HCl in order to protect the integrity of the test compound.

### DETAILED DESCRIPTION OF THE INVENTION

Except where otherwise stated, R, R¹, R² in the reaction schemes and discussions that follow are defined as above. Unless otherwise stated reaction conditions for all reaction schemes were conducted under ambient atmospheric conditions.

Scheme 1 refers to a process of the present invention for the preparation of a 2,2,4-trimethyl-1,5-benzodiazepine from the corresponding aromatic ortho-diamine.

In Scheme 1 the 2,2,4-trimethyl-1,5-benzodiazepine is prepared from an aromatic ortho diamine or a mixture containing a residue of said aromatic ortho-diamine by treating said diamine or mixture with at least 2 molar equivalents of acetone, an excess of acetone, or a large excess of acetone, preferably a large excess of acetone, under nearly neutral conditions, in the presence of a catalytic amount of strong acid, preferably HCl at about 20° C. to about 25° C.

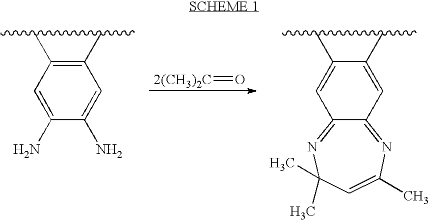

The process of Scheme 1 is followed by quantitative HPLC.

Scheme 2 refers to the preparation of the compound of formula I from the compound of formula II in substantially pure form or from a mixture containing the compound of formula II as a residue, said residue, for example, remaining in the reaction product after treatment of the compound of formula II with a cyclizing agent. Such cyclizing reactions are described in Coe, et al, U.S. Pat. No. 6,410,550 B1 which is incorporated herein by reference in its entirety. To avoid further reaction with the cyclizing agent during analysis which could lead to an underestimate of the aromatic ortho diamine residue level, the sample is derivatized by treatment with acetone. In general, an aliquot of the reaction product resulting from treatment of the compound of formula II with a cyclizing agent is treated with acetone containing a catalytic amount of strong acid, preferably hydrochloric acid at about room temperature to produce the 2,2,4-trimethyl-1,5-benzodiazepine of formula I and then diluted and dissolved with a specified volume of a reaction inert solvent such as acetonitrile. In the process of the present invention, overly acidic or overly basic conditions could compromise the structure of the test compound thereby making analysis confusing. The use of acetone is preferred for the formation of derivatives, as it minimizes potential side reactions, and is much more environmentally friendly than other conventional derivatizing agents. A large excess of acetone can be used, thus achieving much higher levels of derivatization than with the conventional reagents discussed above, and acetone is compatible with the analytical assay methodology.

The quantity of the 2,2,4-trimethyl-1,5-benzodiazepine derivative of formula I can then be determined by any analytical method known in the art capable of distinguishing the compound of formula I from the product of the reaction of the compound of formula II with the cyclizing agent, preferably a chromatographic method, most preferably analytical HPLC. The quantity of the compound of formula II originally in the reaction mixture is then determined by comparison with a standard curve obtained by derivatizing known quantities of the compound of formula II with acetone. When R is trifluoroacetyl in the compound of formula I and the compound of formula II the analysis will provide the quantity of 1-(4,5-Diamino-10-azatricyclo[6.3.1.02,7]dodeca-2(7),3,5-triene-10-yl)-2,2,2-trifluoroethanone in the analyte.

Scheme 2 also refers to the preparation of a compound of formula I from a compound of formula II by treatment with acetone, preferably a large excess of acetone, in the presence of a catalytic amount of a strong acid, preferably hydrochloric acid, at about room temperature, or in the absence of acid at about the reflux temperature of acetone, preferably in the absence of acid at about the reflux temperature of acetone.

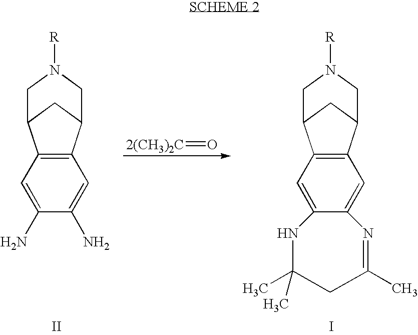

Scheme 3 refers to the preparation of a compound of formula III from the compound of formula Ia, wherein R² is a hydrolyzable protective group, as defined above for R², preferably trifluoroacetyl, by treating the compound of formula Ia with an acidic or basic hydrolyzing agent to remove protective group R². When the protective group is trifluoroacetyl or the like hydrolysis is accomplished with an excess of aqueous basic hydrolyzing agent such NaOH, KOH or LiOH, preferably about 4 molar equivalents of NaOH. Those skilled in the art will recognize that other means such as reduction and catalytic hydrogenation may also be used to remove protective groups from a compound of the formula I or Ia. The compound of formula III may also be prepared, for example, by removal of a benzylic protective group from a compound of the formula I by catalytic hydrogenation using methods well known in the art.

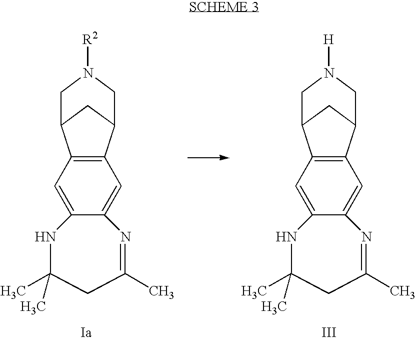

### EXAMPLES

### Example 1

### 7,11-Methanoazepino [4,5-h]-1,5-benzodiazepine. 1,2,3,7,8,9,10,11-octahydro-2,2,4-trimethyl-9-(trifluoroacetyl)

1-(4,5-Diamino-10-azatricyclo[6.3.1.02,7]dodeca-2(7),3,5-triene-10-yl)-2,2,2-trifluoroethanone was dissolved in acetone (50 ml) and warmed to reflux for 2 hours. Reaction progress was monitored by TLC (7:3 EtOAc:Hexanes as eluent). The disappearance of the 1-(4,5-Diamino-10-azatricyclo[6.3.1.02,7]dodeca-2(7),3,5-triene-10-yl)-2,2,2-trifluoroethanone spot was observed along with the emergence of a more polar spot, indicating product formation. A faint amount of starting material was observed along with an unidentified less polar spot when the reaction was stopped. The orange solution was vacuum distilled to produce a sticky oil. The sticky oil was taken up in CH₂Cl₂, silica gel (15 g) was added and the material adsorbed onto the silica gel. The material was loaded onto a chromatography column (7:3 EtOAc:Hexanes to pack the column), and eluted with straight EtOAc.

Clean fractions containing only the desired acetone adduct were combined and vacuum distilled to an oil. Vacuum drying overnight at 45° C. afforded 2.5 g of a tan solid (49.0%). Analytical Data: HPLC 99.5% pure by area; ¹H NMR (d₆ DMSO): δ=6.77 (1H, d), 6.69 (1H, d), 4.47 (1H, d), 4.04 (1H, d), 3.63 (1H, m), 3.56 (1H, m), 3.35 (2H, s), 3.06-3.17 (3H, m), 2.17 (3H, s),1.95-2.04 (2H, m), 1.19 (6H,d); MS (m+1) 366; mp 168-169° C.

### Example 2

### 7,11-Methanoazepino [4,5-h]-1,5-benzodiazepine, 1,2,3,7,8,9,10,11-octahydro-2,2,4-trimethyl

7,11-Methanoazepino [4,5-h]-1,5-benzodiazepine, 1,2,3,7,8,9,10,11-octahydro-2,2,4-trimethyl-9-(trifluoroacetyl)-(405 mg) was added to toluene (3 ml) and gently warmed to 35° C. to facilitate dissolution. In a separate vessel, NaOH (177 mg, 4.0 equivalents) was dissolved in H₂O (2 ml) and added to the warm toluene solution. The biphasic mixture was warmed to 40° C. for 3 hours, slowly cooled to room temperature, then allowed to stir overnight. Reaction progress was monitored by TLC (9: 1 CH₂Cl₂: MeOH) and showed the disappearance of the starting material and the emergence of a much more polar spot identified as the desired deprotected product.

The biphasic mixture was diluted with toluene (20 ml) stirred 15 min., transferred to a separatory funnel and the phases separated. The aqueous layer was extracted with toluene (10 ml), and the toluene layers combined. The toluene solution was stirred with both Na₂SO₄ and activated charcoal, filtered, and vacuum distilled to an oil. Drying in a vacuum oven overnight at 45° C. afforded 193 mg (64.5%) of a pale orange crusty solid. HPLC: 95.8% purity by area; ¹H NMR (CDCl₃): δ=6.94 (1H, s), 6.58 (1H, s), 2.86-2.94 (5H, m), 2.63 (2H, d), 2.35 (3H, s), 2.21 (2H, m), 1.90 (1H, d), 1.83 (2H, m), 1.37 (3H, s), 1.31 (3H, s); MS (m+1): δ=270; mp: 63-65° C.

### Example 3

### Determination of 1-(4,5-Diamino-10-azatricyclo[6.3.1.0^{2,7}]dodeca-2(7),3,5-triene-10-yl)-2,2,2-trifluoroethanone Residue in 1-(5,8,14-Triazatetracyclo[10.3.1.0^{2,11}.0^{4,9}]hexadeca-2(11),3,5,7,9-pentaene-14-yl)-2,2,2-trifluoroethanone (Reaction Product With Glyoxal)

### Preparation of Standards

Derivatizing Agent: 300 mg of 37% HCl is dissolved in 50 mL HPLC grade Acetone.

Stock Standard Solution:—A 10 mg sample of 1-(4,5-Diamino-10-azatricyclo[6.3.1.02,7]dodeca-2(7),3,5-triene-10-yl)-2,2,2-trifluoroethanone is weighed and transferred to a 100 mL volumetric flask. A 10 mL aliquot of derivatizing agent is added and the mixture is stirred with a micro-stir bar at room temperature for 15 minutes. The solution is then brought to volume with acetonitrile. External standards are prepared from the stock solution using acetonitrile as a solvent. Standards are prepared and analyzed from 1 ppm to 100 ppm based on a 30 mg/mL sample concentration.

### Sample Preparation

About 300 mg of 1-(5,8,14-Triazatetracyclo[10.3.1.0^{2,11}.0^{4,9}]hexadeca-2(11),3,5,7,9-pentaene-14-yl)-2,2,2-trifluoroethanone is weighed and transferred to a 10 mL volumetric flask. A 1 mL aliquot of derivatizing agent is added to the flask and the slurry is swirled rigorously. Acetonitrile is then used to dissolve the slurry and bring the solution to volume.

Chromatography:

Mobile Phase A: Water/TFA 2000/2 (v/v) pH 3.0 with NH₄O H

Mobile Phase B: Water/Acetonitrile/TFA 200/1800/2 (v/v/v); pH 3.0 with
NH₄OH

Column: Waters Symmetry C18, 4.6×250 cm, or equivalent

Run Time: 30 minutes

Flow Rate: 1.0 mL/min

DAD: λ=230 nm

Inj. Vol.: 50.0 μL

Column Temp: 40° C.

Gradient:

| Time (min) | % A | % B | Flow (ml/min) |
|---|---|---|---|
| 0.0 | 70 | 30 | 1.0 |
| 2.5 | 70 | 30 | 1.0 |
| 22.0 | 30 | 70 | 1.0 |
| 23.0 | 70 | 30 | 1.0 |
| 30.0 | 70 | 30 | 1.0 |

MSD Conditions: (Electrospray)

Signal 1 (POSITIVE):

| Time(min) | GroupName | SIM Ion | Fragmentor | Gain EMV |
|---|---|---|---|---|
| 0.00 | Group 1 | 366.10 | 120 | 1.0 |
| 12.00* | Not Active | | | |

*Sample stream must be diverted to waste prior to elution of main band 1-(5,8,14-Triazatetracyclo[10.3.1.0^{2,11}.0^{4,9}]hexadeca-2(11),3,5,7,9-pentaene-14-yl)-2,2,2-trifluoroethanone

| | Retention |
|---|---|
| Compound | Time (min.) |
| 7,11-Methanoazepino [4,5-h]-1,5-benzodiazepine, | 10 |
| 1,2,3,7,8,9,10,11-octahydro-2,2,4-trimethyl-9- | |
| (trifluoroacetyl)-1-(5,8,14- | |
| Triazatetracyclo[10.3.1.0^{2,11}.0^{4,9}]hexadeca- | 13 |
| 2(11),3,5,7,9-pentaene-14-yl)-2,2,2-trifluoroethanone | |

Generation of Correlation Curve for:

1-(4,5-Diamino-10-azatricyclo[6.3.1.02,7]dodeca-2(7),3,5-triene-10-yl)-2,2,2-trifluoroethanone

Linearity

| | |
|---|---|
| STD WT (mg) | 10.49 |
| STD Potency | 0.991 |
| Stock STD (mg/mL) | 0.1040 |

| STD | PPM | mg/mL | Area |
|---|---|---|---|
| A | 100 | 0.0020791 | 2.665E+06 |
| B | 50 | 0.0010396 | 1.406E+06 |
| C | 20 | 0.0004158 | 5.769E+05 |
| D | 10 | 0.0002079 | 2.990E+05 |
| E | 5 | 0.0001040 | 1.495E+05 |
| F | 1 | 0.0000208 | 3.072E+04 |
| G | 0.5 | 0.0000104 | 1.543E+04 |
| H | 0.1 | 0.0000021 | 3.023E+03 |

[image]

What is claimed is:

1. A method for the analytical determination of an aromatic ortho diamine contaminant in a pharmaceutical compound or intermediate thereof, comprising the steps of: (i) treating the pharmaceutical compound or intermediate thereof containing the contaminant with acetone to form a 2,2,4-trimethyl-1,5-benzodiazepine derivative of the aromatic ortho diamine contaminant, (ii) determining the quantity of the 2,2,4-trimethyl-1,5-benzodiazepine derivative in the pharmaceutical compound or intermediate thereof, and (iii) correlating the quantity of the 2,2,4-trimethyl-1,5-benzodiazepine derivative with the quantity of aromatic ortho diamine contaminant originally present in the pharmaceutical compound or intermediate thereof.

2. The method of claim 1 wherein the pharmaceutical compound or intermediate thereof contains residues of a cyclizing agent.

3. The method of claim 1 wherein the aromatic ortho diamine contaminant has the formula 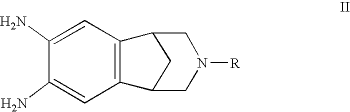
wherein R is H, COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 fluoro or chloro atoms; benzyl or t-butoxycarbonyl (t-Boc);
and the 2,2,4-trimethyl-1,5-benzodiazepine derivative formed in step (i) has the formula 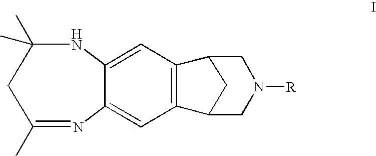
wherein R is H, COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 fluoro or chloro atoms; benzyl or t-butoxycarbonyl (t-Boc).

4. The method of claim 3 wherein R is —C(═)CF₃ in the compound of formula II and the compound of formula I.

5. The method of claim 4 wherein step (i) is conducted in the presence of a catalytic amount of strong acid.

6. The method of claim 5 wherein the acid is HCl.

7. The method of claim 1 wherein step (ii) the quantity of the 2,2,4-trimethyl-1,5-benzodiazepine derivative is determined by HPLC.

8. A compound of the formula 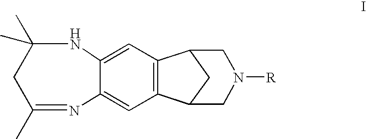
wherein R is H, COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 fluoro or chloro atoms; benzyl or t-butoxycarbonyl (t-Boc).

9. The compound of claim 8 wherein R is H.

10. A process for the preparation of a compound of formula 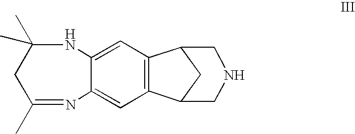
wherein a compound of the formula 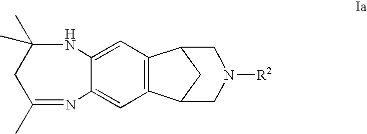
wherein R² is a protective group selected from COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 fluoro or chloro atoms; or t-butoxycarbonyl (t-Boc), is treated with an aqueous base.

11. A process for the preparation of a compound of formula 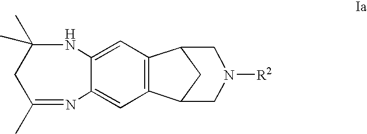
wherein R² is a protective group selected from COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 fluoro or chloro atoms; or t-butoxycarbonyl (t-Boc),
wherein a compound of the formula 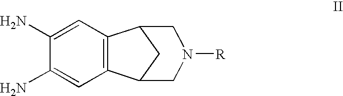
wherein R is H, COOR¹ wherein R¹ is (C₁-C₆)alkyl, allyl, 2,2,2-trichloroethyl or (C₁-C₆)alkyl; —C(═O)H, —C(═O)(C₁-C₆)alkyl wherein the alkyl moiety may optionally be substituted with from 1 to 3 fluoro or chloro atoms; or t-butoxycarbonyl (t-Boc); is treated with at least 2 molar equivalents of acetone.
